(19) United States
(12) Patent Application Publication
(10) Pub. No.: US 2011/0190464 A1
(43) Pub. Date: Aug. 4, 2011

(54) METHOD FOR THE SYNTHESIS OF BIORESOURCED ACRYLIC ACID ESTERS

(75) Inventors: Jean-Luc Dubois, Millery (FR); Alain Riondel, Forbach (FR)

(73) Assignee: Arkema France, Colombes (FR)

(21) Appl. No.: 13/055,864

(22) PCT Filed: Jul. 24, 2009
(86) PCT No.: PCT/FR2009/051491

§ 371 Date: Apr. 4, 2011

(30) Foreign Application Priority Data
Jul. 25, 2008 (FR) 0855125

(51) Int. Cl. C08F 20/06; C07C 67/30

(52) U.S. Cl. 526/328; 560/214

(57) ABSTRACT

The present invention relates to a method for the synthesis of an acrylic acid ester of formula CH₂═CH—COOR, where R is an alkyl radical having between 1 and 18 carbon atoms and optionally where one of the carbon atoms in the alkyl radical may be replaced with a nitrogen atom. In an embodiment of the invention, glycerol is subjected to a dehydration reaction in the presence of an acid catalyst to obtain acrolein. The acrolein formed is transformed by catalytic oxidation into acrylic acid, which is subjected to an esterification reaction by means of an alcohol of the formula ROH in which R has the meaning as above. The invention also relates to bioresourced esters produced according to the method, and to synthesized polymers using the esters of the invention as polymerization monomers or comonomers.

The invention relates to a process for the synthesis of acrylic acid esters of formula CH₂═CH—COO—R in which R represents a linear or branched alkyl radical comprising from 1 to 18 carbon atoms and comprising, if appropriate, a heteroatom, such as nitrogen. Acrylic acid esters, acrylates, are widely used industrially. The range of uses for the manufacture of polymers is broad. However, some of them require the acrylate used as monomer or as comonomer in the manufacture of copolymers or terpolymers to adhere to standards as regards purity. These standards of purity with regard to certain compounds are specific and directly related to the polymer of the final application. It is difficult to achieve these standards without resorting to very expensive fractionation and purification techniques.

Acrylates are prepared from acrylic acid either by simple esterification or by a transesterification reaction of a light acrylate of methyl acrylate, ethyl acrylate, propyl acrylate or butyl acrylate type with the hydroxylated compound necessary for the synthesis of the polymer constituting or participating in the structure of the final ester.

By way of example, the ester 2-ethylhexyl acrylate of formula CH₂═CH—COO—CH₂—CH (C₂H₅)—(CH₂)₃—CH₃, normally referred to as 2EHA, is generally obtained by direct esterification of acrylic acid of formula CH₂═CH—COOH with 2-ethylhexanol according to the following reaction:

CH₂═CH—COOH+CH₃—(CH₂)₃—CH(C₂H₅)—CH₂OH→CH₂═CH—COO—CH₂—CH(C₂H₅)—(CH₂)₃—CH₃+H₂O

For its part, the aminoester of formula CH₂═CH—COO—CH₂—CH₂—N (CH₃)₂, dimethylaminoethyl acrylate, normally referred to as ADAME, is generally obtained by transesterification of the acrylic ester of formula CH₂═CH—COOR₀ according to the following reaction:

CH₂═CH—COOR₀+(CH₃)₂N—CH₂—CH₂OH→CH₂═CH—COO—CH₂—CH₂—N(CH₃)₂+R₀OH

R₀ being either CH₃ or C₂H₅ or C₃H₇ or C₄H₉.

Butyl acrylate (BuA), an ester of formula CH₂═CH—COO—C₄H₉, very often used in copolymerization processes in order to confer an elastomeric nature on the copolymer, is generally synthesized by direct esterification of acrylic acid with n-butanol.

Methylacrylate (MA), of formula CH₂═CH—COO—CH₃, which is very often used in copolymerization processes to manufacture fibers, is generally synthesized by direct esterification of acrylic acid with methanol.

Ethyl acrylate (EA), of formula CH₂═CH—COO—C₂H₅, which is very often used in copolymerization processes in order to confer cohesion on textile fibers, is generally synthesized by direct esterification of acrylic acid with ethanol.

It is often difficult to obtain these monomers with a degree of purity which is satisfactory for the final industrial application.

Mention may be made, on this subject, of French Patent No. 2 777 561 on behalf of the Applicant Company, which describes a particularly sophisticated process for the manufacture of ADAME which makes it possible to obtain a product having contents of “contaminants”, such as ethyl acrylate (EA) and dimethylaminoethanol (DMAE), lower than strict thresholds.

As regards the synthesis of 2EHA, which is catalyzed by the acid route, use is made industrially of heterogeneous catalysis employing acid resins. These are generally strong cationic resins of sulfonic type. The problem posed by the manufacture of 2EHA is the presence in the ester produced of a high level of impurities and in particular of compounds of maleic type which take the ester outside the specifications allowed for the sale of the ester in the majority of fields, in particular that of pressure-sensitive adhesives (PSA).

The acrylic acid (AA) employed as starting material in this type of process is essentially produced industrially from propylene. The latter is subjected to a two-stage oxidation according to the following reaction process:

CH₂═CH—CH₃+O₂→CH₂═CH—CHO+H₂O 2 CH₂═CH—CHO+O₂→2 CH₂═CH—COOH,

i.e. an overall reaction:

CH₂═CH—CH₃+3/2O₂→CH₂═CH—COOH+H₂O.

This synthesis of acrylic acid is known as “petrochemical synthesis” and thus uses, as starting material, propylene subjected to two successive oxidations. It exhibits the advantage of making possible the synthesis either of acrolein (ACO), which is sold as is, if the synthesis is halted at the first stage, or of acrylic acid, if the oxidation is pushed to the end.

However, this highly effective oxidation process exhibits the disadvantage of forming byproducts or impurities, such as, in particular, furfural, cyclic aldehyde, maleic anhydride or maleic acid, when it is very difficult to separate from the main product, even after the entire conventional purification process.

In the case of the manufacture of acrylic acid, this reaction is generally carried out in the vapor phase, generally in two stages, which can be carried out in two separate reactors or just one reactor:
the first stage carries out the substantially quantitative oxidation of the propylene to give a mixture rich in acrolein (ACO) in which AA is a minor component,
the second stage completes the conversion of the ACO to AA.

The gas mixture resulting from the oxidation reaction 2nd stage is composed, apart from the acrylic acid:
of light compounds which are noncondensable under the temperature and pressure conditions generally employed (nitrogen, unconverted oxygen and propylene, propane present in the propylene reactant, carbon monoxide and carbon dioxide formed in a small amount by final oxidation),
of condensable light compounds: in particular water, generated by the propylene oxidation reaction, unconverted acrolein, light aldehydes, such as formaldehyde and acetaldehyde, and acetic acid, the main impurity generated in the reaction section,
of heavy compounds: furfuraldehyde, benzaldehyde, maleic anhydride, benzoic acid, and the like.

The second phase of the manufacture consists in recovering the AA from the gas mixture resulting from the 2nd stage by introducing this gas at the bottom of an absorption column, where it encounters, countercurrentwise, a solvent introduced at the column top. In the majority of the processes described, the solvent employed in this column is water or a hydrophobic solvent with a high boiling point.

In the case of absorption processes using water as absorbent solvent, the additional purification stages comprise a stage of dehydration, generally carried out in the presence of a water-immiscible solvent in an extraction or heteroazeotropic distillation column, then a stage of removal of the light products, in particular acetic acid and formic acid, and a stage of separation of the heavy compounds.

In the case of processes using a hydrophobic solvent, the stages are essentially the same, except for the removal of water, which is carried out at the top of the first absorption column. These processes exhibit the main disadvantages of employing a very large amount of solvent with a high boiling point which, in addition to the cost of the operation, can cause problems of discharge of product which is harmful to the environment and of polymerization in the columns promoted by the high levels of heat imposed by the solvent at the column base.

In these processes, beyond what has just been mentioned, the separation of the heavy compounds constitutes the main problem.

Furthermore, this process exhibits the disadvantage of using propylene, a fossil starting material resulting from oil. It is known that oil will eventually disappear and that, in any case, it will become increasingly expensive.

It has been found, for example, that furfural, even present in the form of traces in the acrylic acid, i.e. at a concentration of greater than 0.01% by weight, can, in some subsequent conversions, exhibit major disadvantages by having a strong negative effect on the degree of polymerization required for the product in the application envisaged. Similarly, it has been observed that this process also exhibits the disadvantage of synthesizing, as byproduct, maleic anhydride or maleic acid which, at a concentration of greater than 0.1% by weight, can, in some applications, constitute a major disadvantage because of the acidity generated in the monomer.

As regards BuA, the presence of the iso isomer, isobutyl acrylate, can modify the Tg (glass transition temperature) of the final polymers.

As regards EA, furfuraldehyde constitutes an impurity which is harmful to the manufacture of ADAME and the subsequent use of this monomer as cationic flocculant precursor.

It is an object of the invention to overcome these disadvantages by providing a novel method of synthesis of these esters employing another process for the synthesis of acrylic acid, the subject of more recent developments, using glycerol instead of propylene as starting material. Furthermore, the use of alcohols, themselves of vegetable and/or animal origin, will make it possible to strengthen the “bioresourced” nature of the process by essentially consuming renewable starting materials.

The process for the synthesis of acrylic acid by this route is a two-stage process consisting, in a first stage, in dehydrating the glycerol to give acrolein and then, in a second stage, in oxidizing the acrolein to give acrylic acid, according to the following reaction process:

CH₂OH—CHOH—CH₂OH⇄CH₂═CH—CHO+2H₂O CH₂═CH—CHO+½ O₂→CH₂═CH—COOH.

It has been known for a long time that glycerol can lead to the preparation of acrolein. Glycerol (also known as glycerin) results from the methanolysis of oils of vegetable and/or animal origin at the same time as the methyl esters, which are themselves employed in particular as fuels in gas oil and domestic heating oil. Glycerol can also derive from hydrolysis of vegetable and/or animal oils, resulting in the formation of fatty acids, or from the saponification of vegetable and/or animal oils, resulting in the formation of soaps. This is a natural product which enjoys a “green” aura, it is available in large amounts and it can be stored and transported without difficulty. Numerous studies have been devoted to enhancing glycerol in value according to its degree of purity, and the dehydration of glycerol to give acrolein is one of the routes envisaged.

The reaction mentioned above, deployed in order to obtain acrolein from glycerol, is an equilibrium reaction. As a general rule, the hydration reaction is favored at low temperatures and the dehydration is favored at high temperatures. In order to obtain acrolein, it is thus necessary to employ a satisfactory temperature and/or a partial vacuum in order to displace the reaction. The reaction can be carried out in the liquid phase or in the gas phase. This type of reaction is known to be catalyzed by acids. The reaction for the oxidation of acrolein is normally carried out in the gas phase in the presence of an oxidation catalyst.

In order to illustrate the studies carried out for decades on this subject, mention may be made of French Patent No. 69.5931, in which, in order to obtain acrolein, glycerol vapors are passed at high temperature over acid salts (phosphoric acid salts). The yields shown are greater than 75% after fractional distillation. In U.S. Pat. No. 2,558,520, the dehydration reaction is carried out in the gas/liquid phase in the presence of diatomaceous earths impregnated with phosphoric acid salts in suspension in an aromatic solvent. A degree of conversion of the glycerol to give acrolein of 72.3% is obtained under these conditions.

More recently, U.S. Pat. No. 5,387,720 describes a process for the production of acrolein by dehydration of glycerol in the liquid phase or in the gas phase over solid acid catalysts defined by their Hammett acidity. According to this patent, an aqueous solution comprising from 10 to 40% of glycerol is used and the reaction is carried out at temperatures of between 180° C. and 340° C. in the liquid phase and between 250° C. and 340° C. in the gas phase. According to the authors of this patent, the gas-phase reaction is preferable as it makes it possible to have a degree of conversion of the glycerol of approximately 100%. This reaction results, after condensation, in an aqueous acrolein solution comprising byproducts, such as hydroxypropanone, propionaldehyde, acetaldehyde, acetone, addition products of acrolein with glycerol, and the like. A proportion of approximately 10% of the glycerol is converted to hydroxypropanone, which is encountered as predominant byproduct in the acrolein solution. The acrolein is recovered and purified by fractional condensation or distillation. For a liquid-phase reaction, a conversion of 15-25% cannot be exceeded without the risk of forming an unacceptable amount of byproducts and of obtaining a quality of monomer (acrolein or acrylic acid) incompatible with the desired quality. In the document WO 06/087083, the reaction for the dehydration of glycerol in the gas phase is carried out in the presence of molecular oxygen.

The document WO 06/087084 recommends the use of highly acidic solid catalysts having a Hammett acidity H₀ of between −9 and −18 for the dehydration of glycerol in the gas phase. In general, the glycerol used as starting material for the dehydration reaction is an aqueous solution.

In order to manufacture the acrylic acid, the acrolein is subjected, in a second stage, to an oxidation. In Patent Application EP 1 710 227, the reaction product resulting from the reaction for the dehydration of glycerol in the gas phase is subjected to a subsequent stage of oxidation in the gas phase in order to obtain acrylic acid. The process is carried out in two reactors in series, each comprising a catalyst suitable for the reaction carried out. Application WO 06/092272 describes the entire process with its first two stages, dehydration and oxidation, followed by additional stages in order to obtain the purified acrylic acid.

A preferred alternative form of the process comprising two stages, described in Patent Application No. FR 2 909 999 of 19 Dec. 2006, consists in carrying out the partial condensation of the water in the reaction gases resulting from the first stage of dehydration of the glycerol, before introducing the gas into the reactor of the 2nd stage of oxidation to give acrylic acid. This additional condensation stage consists in cooling the gas stream to a temperature such that a portion of the water is condensed as liquid phase and all of the acrolein remains in the gaseous form.

The proposal has also been made to carry out the reaction in just one stage. Application WO 06/114506 describes a process for the preparation of acrylic acid in one stage by an oxydehydration reaction on the glycerol in the presence of molecular oxygen with the 2 consecutive dehydration and oxidation reactions.

It is an object of the invention to overcome the abovementioned disadvantages by providing, in order to manufacture esters, for the use of an acrylic acid obtained by a different method of synthesis using glycerol as main starting material.

A subject matter of the present invention is a process for the synthesis of an acrylic acid ester of formula CH₂═CH—COO—R in which R represents a linear or branched alkyl radical comprising from 1 to 18 carbon atoms and comprising, if appropriate, a heteroatom, nitrogen, characterized in that, in a first stage, glycerol CH₂OH—CHOH—CH₂OH is subjected to a dehydration reaction in the presence of an acid catalyst, in order to obtain acrolein of formula CH₂═CH—CHO, then, in a second stage, the acrolein thus formed is converted by catalytic oxidation to give acrylic acid CH₂═CH—COOH and then, in a third stage, the acid resulting from the second stage is subjected to a reaction for esterification by means of an alcohol ROH in which R has the meaning given above.

In an alternative form of the process, the third stage is carried out in two substages, the first consisting in esterifying the acrylic acid with a light alcohol comprising from 1 to 4 carbon atoms and then, in the second, in converting the ester of the light alcohol chosen, generally methyl or ethyl ester, to the desired ester by transesterification with the alcohol ROH. This alternative form applies in particular to the case where the alcohol ROH comprises a heteroatom, such as nitrogen.

In another alternative form of the process, the first two stages can be carried out, as was described in Application WO 06/114506, in a single reactor by an oxydehydration reaction of the glycerol in the presence of molecular oxygen employing the two consecutive dehydration and oxidation reactions.

In another alternative form of the process, an intermediate stage of condensation of the water present in the stream resulting from the first stage of dehydration of the glycerol is carried out, before introducing into the reactor for the 2nd stage of oxidation to give acrylic acid.

The first stage of dehydration of the glycerol is carried out in the gas phase in the reactor in the presence of a catalyst at a temperature ranging from 150° C. to 500° C., preferably of between 250° C. and 350° C., and a pressure of between 10⁵ and 5×10⁵ Pa.

The reactor used can operate as a fixed bed, as a fluidized bed or as a circulating fluidized bed or in a configuration as modules (sheets or pans) in the presence of solid acid catalysts.

The catalysts which are suitable are homogeneous or multiphase materials which are insoluble in the reaction medium and which have a Hammett acidity, denoted H₀, of less than +2, as indicated in U.S. Pat. No. 5,387,720, which refers to the paper by K. Tanabe et al. in “Studies in Surface Science and Catalysis”, vol. 51, 1989, chap. 1 and 2; the Hammett acidity is determined by amine titration using indicators or by adsorption of a base in the gas phase. The catalysts meeting the criterion of H₀ acidity of less than +2 can be chosen from natural or synthetic siliceous materials or acidic zeolites; inorganic supports, such as oxides, covered with mono-, di-, tri- or polyacidic inorganic acids; oxides or mixed oxides or also heteropolyacids.

These catalysts can generally be composed of a heteropolyacid salt in which the protons of the said heteropolyacid are exchanged with at least one cation chosen from elements belonging to Groups I to XVI of the Periodic Table of the Elements, these heteropolyacid salts comprising at least one element chosen from the group consisting of W, Mo and V.

Mention may also be made, among mixed oxides, of those based on iron and on phosphorus and of those based on cesium, phosphorus and tungsten.

The catalysts are advantageously chosen from zeolites, Nafion® composites (based on sulfonic acid of fluoropolymers), chlorinated aluminas, phosphotungstic and/or silicotungstic acids and acid salts, and various solids of the type comprising metal oxides, such as tantalum oxide Ta₂O₅, niobium oxide Nb₂O₅, alumina Al₂O₃, titanium oxide TiO₂, zirconia ZrO₂, tin oxide SnO₂, silica SiO₂ or silicoaluminate SiO₂/Al₂O₃, impregnated with acid functional groups, such as borate BO₃, sulfate SO₄, tungstate NO₃, phosphate PO₄, silicate SiO₂ or molybdate MoO₃ functional groups, and the like. According to the literature data, these catalysts all have a Hammett acidity H₀ of less than +2.

The preceding catalysts can additionally comprise a promoter, such as Au, Ag, Cu, Pt, Rh, Pd, Ru, Sm, Ce, Yt, Sc, La, Zn, Mg, Fe, Co, Ni or montmorillonite.

The preferred catalysts are phosphated zirconias, tungstated zirconias, silica zirconias, titanium or tin oxides impregnated with tungstate or phosphotungstate, phosphated aluminas or silicas, heteropolyacids or heteropolyacid salts, iron phosphates and iron phosphates comprising a promoter.

The second stage of the process according to the invention is carried out under the following conditions.

The reaction for the oxidation of the acrolein-rich stream generated during the first stage (acrolein concentration generally of 2 to 15% by volume) is carried out in the presence of molecular oxygen, which can equally be introduced in the form of air or in the form of air enriched or diluted in molecular oxygen, at a content ranging from 1 (minimum stoichiometry for a concentration of ACO of 2% of the reactor inlet) to 20% by volume, with respect to the incoming stream, and in the presence of gases which are inert under the reaction conditions, such as N₂, CO₂, methane, ethane, propane or other light alkanes, and of water. The inert gases necessary for the process, in order to prevent the reaction mixture from lying within the flammability region, can optionally be composed, in all or part, of the gases obtained at the top of the separation column placed downstream of the second stage reactor.

The oxidation reaction is carried out at a temperature ranging from 200° C. to 350° C., preferably from 250° C. to 320° C., and under a pressure ranging from 10⁵ to 5×10⁵ Pa.

Use is made, as oxidation catalyst, of all types of catalysts well known to a person skilled in the art for this reaction. Use is generally made of solids comprising at least one element chosen from the list Mo, V, W, Re, Cr, Mn, Fe, Co, Ni, Cu, Zn, Sn, Te, Sb, Si, Pt, Pd, Ru and Rh, present in the metallic form or in the oxide, sulfate or phosphate form. Use is made in particular of the formulations comprising, in the form of mixed oxides, Mo and/or V and/or W and/or Cu and/or Sb and/or Fe as main constituents.

The reactor can operate as a fixed bed, as a fluidized bed or as a circulating fluidized bed. It is also possible to use a plate exchanger with a modular arrangement of the catalyst, such as those described in the patents mentioned below: EP 995 491, EP 1 147 807 or US 2005/0020851.

The third stage of esterification carried out in order to synthesize the esters, such as ethyl acrylate, methyl acrylate, butyl acrylate, propyl acrylate and 2-ethylhexyl acrylate, is carried out under the following conventional conditions.

The catalytic reaction is carried out under the following temperature and pressure conditions: temperature from 60 to 90° C. and pressure from 1.2×10⁵ Pa to 2×10⁵ Pa.

The catalysts of the esterification reaction are acids. They can be chosen from inorganic acids, such as sulfuric acid, sulfonic or phosphoric acids or p-toluenesulfonic, benzenesulfonic, methanesulfonic or dodecylsulfonic derivatives, and the like, the reaction taking place in a homogeneous single-phase medium. The catalysts can also be solid polymers (ion-exchange resins having an acidic nature) and, in this case, the reaction takes place in a heterogeneous two-phase medium.

The latter catalysts will generally be sulfonated styrene/divinylbenzene (DVB) copolymers known as “acidic resins” of gel or macroporous type, the DVB content of which can vary from 2 to 25% by weight and the acidity of which, expressed as H⁺ eq./1 of resins, is between 1 and 2.

They are, for example, supplied by Lanxess under the name Lewatit or by Röhm and Haas under the name Amberlyst.

The catalysts used are preferably acidic ion-exchange resins of Amberlyst 131 and Lewatit K1461 type.

The reaction is carried out in a reactor which operates continuously.

For the alternative embodiment of the process employing an esterification with a light alcohol as described above, followed by a transesterification with the “target” alcohol for the desired ester, the conditions of the transesterification are as follows.

The transesterification reaction is carried out batchwise or continuously, as described in Patents FR 2 617 840, FR 2 777 561 and FR 2 876 375.

The transesterification process consists in reacting, while bubbling with air, in the presence of a catalyst and of at least one polymerization inhibitor, at a temperature of between 20 and 120° C. and at a pressure equal to atmospheric pressure or lower than atmospheric pressure, the light acrylic ester with the target alcohol, generally a dialkylaminoalcohol, in a light acrylic ester to aminoalcohol molar ratio of between 1.3 and 5, in the presence of a catalyst and, during the reaction, in withdrawing the light ester/light alcohol azeotropic mixture and, at the end of the reaction, in separating the dialkylaminoalcohol acrylate, generally by distillation.

The term “dialkylaminoalcohol acrylate” is understood to mean dimethylaminoethyl acrylate and diethylaminoethyl acrylate.

Use may be made, as catalysts, of alkyl titanates, such as, for example, ethyl titanate, tin derivatives, such as dibutyltin oxide or distannoxanes, zirconium derivatives, such as zirconium acetylacetonate, magnesium derivatives, such as magnesium ethoxide, or calcium derivatives, such as calcium acetylacetonate. These compounds are involved in a proportion of 10⁻³ to 5×10⁻² mol per mole of dialkylaminoalcohol and preferably in a proportion of 5×10⁻³ to 1×10⁻² mol per mole of dialkylaminoalcohol.

The choice is preferably made of a light acrylic ester to dialkylaminoalcohol molar ratio of between 1.5 and 2.5.

During the reaction, the temperature is preferably maintained between 80 and 120° C. and more preferably between 90 and 115° C. The pressure is preferably maintained between 50 and 85 kPa, that is to say that the reaction is carried out under slightly reduced pressure.

Mention may be made, among dialkylaminoalcohols suitable for the present invention, of diethylaminoethanol and dimethylaminoethanol, with a preference for dimethylaminoethanol.

Use is made, as polymerization inhibitor, of phenothiazine, hydroquinone methyl ether, hydroquinone, di(tert-butyl)methylhydroxytoluene or 4-hydroxy-Tempo, alone or as a mixture, in a proportion of 500 to 2500 ppm with respect to the total charge.

In a preferred embodiment of the process of the present invention, the synthesis is targeted at an acrylic acid aminoester of formula CH₂═CH—COO—CH₂—CH₂—N(CH₃)₂, in which, during the third stage, the acid resulting from the second stage is subjected to an esterification by means of a light alcohol, methyl alcohol or ethyl alcohol, and then, finally, the ester thus formed is subjected to a transesterification reaction by the action of an aminoalcohol of formula (CH₃)₂—N—CH₂—CH₂OH.

The technical problem to be solved is that of achieving the production of acrylic acid esters exhibiting a high level of purity, that is to say, in this case, a content of furfural <3 ppm, which compound is particularly troublesome for the subsequent application of the ester under consideration. This is because these compounds are intended in particular to be converted into quaternary salts, known as “ADAME-Quats”, by the action, for example, of CH₃Cl. These “ADAME-Quats” can participate in the structure of flocculants intended for water treatment in the form of ADAME-Quats/acrylamide copolymers. In point of fact, it has been discovered that the presence in ADAME-Quats of an even very small amount of furfural as impurity has a very strong effect on the degree of polymerization of the monomer, resulting in a molecular weight (Mw) far below that which is necessary for the effectiveness of the product in the application envisaged.

The alternative form of the process consists, in a first stage, in subjecting glycerol to a dehydration reaction in the presence of an acid catalyst having Hammett acidity H₀ of less than +2, then, in a second stage, in oxidizing the acrolein formed to give acrylic acid by oxidation in the presence of a catalyst comprising, in the form of mixed oxides, the following metals Mo and/or V and/or W and/or Cu and/or Sb and/or Fe, then, in a third stage, in esterifying the acid by means of a light alcohol of formula R₀OH in which R₀ represents an alkyl radical comprising from 1 to 4 carbon atoms, preferably ethanol, and, finally, in transesterifying the ester formed by the action of an aminoalcohol of formula (CH₃)₂—N—CH₂—CH₂OH.

On completion of the 3rd stage, the transesterification of the light acrylate by the aminoalcohol of formula (CH₃)₂—N—CH₂—CH₂OH is preferably carried out in the presence of a catalyst composed of tetrabutyl titanate, tetraethyl titanate or tetra(2-ethylhexyl)titanate at a temperature of between 90 and 120° C. in a stirred reactor at a pressure of between 0.5×10⁵ Pa and 10⁵ Pa.

On conclusion of the first stage, the furfural content of the acrolein, after condensation of the water, which represents most of the reaction medium (it should be remembered that the glycerol is treated in the form of an aqueous solution), before it is introduced into the second stage, is of the order of several tens of ppm, to be compared with the several hundred ppm of the outlet stream from the first stage of the propylene process.

This content is subsequently lowered during the subsequent stages of purification of acrylic acid, of esterification and then of transesterification of the ester in order to achieve a slightly lower concentration in the technical AA (TAA), of the order of approximately 10 ppm in the ethyl acrylate and finally of less than 3 ppm in the final ADAME, the effluent resulting from each stage being subjected to purification by distillation. The ADAME obtained is quaternized by the action of methyl chloride, according to the process described, for example, in the documents EP 1 144 356, EP 1 144 357 or WO 00/43348, to result in an aqueous solution ADAMQUAT MC with an active material content of 80%. The ADAMQUAT MC is subsequently polymerized with acrylamide and the polymer obtained is characterized by the measurement of the viscosity at ambient temperature of a molar aqueous NaCl solution comprising 0.1% of the copolymer manufactured, as is illustrated in Patent FR 2 815 036.

In another preferred embodiment of the process of the present invention, the synthesis targets the synthesis of an ester of formula:

CH₂═CH—COO—CH₂—CH(C₂H₅)—(CH₂)₃—CH₃ (2EHA)

with a low content of residual acidity.

The ester of formula CH₂═CH—COO—CH₂—CH(C₂H₅)—(CH₂)₃—CH₃, normally referred to as 2EHA, is generally obtained by esterification of acrylic acid of formula CH₂═CH—COOH with 2-ethylhexanol according to the following reaction:

CH₂═CH—COOH+CH₃—(CH₂)₃—CH(C₂H₅)—CH₂OH⇄CH₂═CH—COO—CH₂—CH(C₂H₅)—(CH₂)₃—CH₃+H₂O.

The equilibrium reaction has to be displaced towards the formation of ester by removing the water, generally by entrainment using a solvent which forms, with the water, a heteroazeotropic mixture or, more simply and preferably, in the form of a mixture composed of the alcohol, the ester and the water, which also forms a heteroazeotropic mixture. After separating by settling, the aqueous phase is removed and the organic phase is recycled to the reaction stage.

The following stages of purification, in order to obtain the pure acrylic ester, consist in removing the light compounds (mainly excess alcohol, unconverted acrylic acid and residual water) at the top of a topping column and in then removing the heavy compounds at the bottom of a tailing column, the pure product being recovered at the top of this column.

The problem posed by the manufacture of the acrylic ester 2EHA from an acrylic acid manufactured according to a conventional process of petrochemical type and then esterified with 2-ethylhexanol by using a process based on acidic resins is the presence in the ester produced of a high level of certain impurities and in particular of compounds of maleic type which take the ester outside the specifications allowed for its sale in the majority of fields, in particular in that of adhesives and of leather and textile treatment, where the presence of an acidity slows down the polymerization process.

The residual acidity of the purified monomer can originate from two main sources: the presence of acrylic acid and the presence of maleic anhydride present as impurity in the acrylic acid used for the esterification. While the removal of the residual acrylic acid can be carried out by removal of the light compounds at the top of the first topping column after the reaction stage, that of the maleic acid or anhydride is much more difficult as maleic anhydride is a compound with a volatility similar to that of 2EHA. The maleic anhydride can originate from the incomplete conversion of this compound to give mono(2-ethylhexyl)maleate and di(2-ethylhexyl)maleate by esterification with the alcohol. This is because mono(2-ethylhexyl)maleate is a compound which is not very stable thermally and which undergoes, in the purification columns, a dismutation to give anhydride and di(2-ethylhexyl)maleate. In the process, the maleic anhydride generated by this dismutation reaction and/or present as impurity in the acrylic acid not converted by esterification cannot be easily removed by distillation, due to its boiling point being similar to that of the monomer, and is responsible for an acidity of the synthesized ester which is harmful to the manufacture of polymers from this ester.

To obtain this product industrially with a satisfactory degree of purity is particularly difficult, and is emphasized in the abovementioned French patent No. 2 818 639.

The only solutions for solving this problem are to remove the maleic anhydride from the charge, which amounts either to using what is referred to as a Glacial Acrylic Acid or to providing an additional stage of removal of the monomaleate which is formed from the maleic anhydride of the charge during the esterification stage, for example by neutralization with sodium hydroxide. Unfortunately, these two solutions are not viable industrially for reasons of additional expenditure.

One of the objects of the invention is to overcome these disadvantages by providing for the use of a novel method of synthesis of 2EHA employing the process for the synthesis of acrylic acid using glycerol instead of propylene as starting material.

The invention targets a process for the synthesis of an acrylic acid ester of formula CH₂═CH—COO—CH₂—CH(C₂H₅)—(CH₂)₃—CH₃, characterized in that, in a first stage, glycerol CH₂OH—CHOH—CH₂OH is subjected to a dehydration reaction in the presence of an acid catalyst in order to obtain acrolein of formula CH₂═CH—CHO, then, in a second stage, the acrolein formed is converted by catalytic oxidation to acrylic acid CH₂═CH—COOH and, finally, in a third stage, the acid resulting from the second stage is subjected to an esterification reaction under acid catalysis with an alcohol of formula CH₃—(CH₂)₃—CH(C₂H₅)—CH₂OH.

The content of maleic anhydride at the outlet of the reactor for the oxidation of acrolein to give AA starting from propylene is of the order of 1% by weight and, after purification up to the stage of “technical” AA (TAA), its content is very generally of the order of 1000 to 1500 ppm. The maleic anhydride present in the TAA will unfortunately remain in the medium during the stage of esterification of the TAA with 2-ethylhexanol to form esters, 2-ethylhexyl monomaleate and predominantly (ten times more) di(2-ethylhexyl)maleate. The separation of the latter, which is a heavy product, is relatively easy by distillation. Unfortunately, it has been found by the Applicant Company that, during the distillation, the monomaleate “dismutates” to give dimaleate, which is easily separated and thus is not a disadvantage, but also to give maleic anhydride, which, for its part, will remain in the 2EHA produced and this at levels far above the thresholds (<40 ppm) of industrial specifications.

The first two stages, dehydration and oxidation, are carried out as described above and the esterification reaction is carried out in the liquid phase at a temperature of between 50 and 150° C. in the presence of a solid acid catalyst, for example of Lewatit K2621 or Amberlyst 15 resin type, under a pressure of between 1 and 3×10⁵ Pa.

One of the main objects of the invention is to use starting materials of natural and renewable origin, that is to say bioresourced starting materials. Independently of the manufacture of acrylic acid from “natural” glycerol, the invention applies to the use, during the esterification, of alcohols ROH of renewable natural origin or resulting from the biomass, in other words bioresourced. If the light alcohols are industrially generally of natural origin, it is otherwise for the higher alcohols. Mention may be made, by way of example, of butanol, which is manufactured by hydroformylation of propylene to give n-butyraldehyde, followed by a hydrogenation to give n-butanol. Apart from the fact that use is still made, in this process, of a fossil starting material, it should be observed that this method of synthesis results in n-butanol comprising traces of isobutanol of the order of 1000 ppm, which all ends up in the butyl acrylate in the form of isobutyl acrylate.

The invention also targets a process for the synthesis of butyl acrylate in which the acrylic acid is manufactured as described above from glycerol and is subsequently esterified with n-butanol obtained by aerobic fermentation of biomass in the presence of bacteria.

The fermentation of renewable materials resulting in the production of butanol, generally with the presence of acetone, is carried out in the presence of one or more appropriate microorganisms. This microorganism may optionally have been modified naturally, by chemical or physical stress, or genetically. Reference is then made to mutant. Conventionally, the microorganism used is a Clostridium; advantageously, it will be Clostridium acetobutylicum or one of its mutants. The lists presented above are not limiting.

The stage of fermentation can also be preceded by a stage of hydrolysis of the starting materials using an enzyme of cellulase type or a complex of several enzymes of cellulase type.

Use may be made, as renewable starting materials, of plant materials, materials of animal origin or materials resulting from recovered materials of plant or animal origin (recycled materials).

Plant materials include in particular sugars, starches and any plant material comprising sugars, cellulose, hemicellulose and/or starches.

Mention may in particular be made, among materials resulting from recovered materials, of plant or organic waste comprising sugars and/or starches and also any fermentable waste.

Advantageously, starting materials of low quality can be used, such as, for example, frost-damaged potatoes, cereals contaminated by mycotoxins or also surpluses of sugar beets, or whey from cheese dairies.

Preferably, the renewable starting materials are plant materials.

The stage of fermentation is generally followed by a stage of isolation of the butanol.

This isolation of butanol consists of a separation of the various reaction products, for example by heteroazeotropic distillation. This separation can also be followed by distillation intended to obtain the butanol in more concentrated form.

A stage for separating the n-butanol from the other isomers may also be provided. Nevertheless, fermentation results in a more restricted number of butanol isomers than the chemical route of hydroformylation of propylene. The analyses of butanol resulting from fermentation of renewable starting materials and of butanol resulting from fossil starting materials are illustrated in the table below.

| | Butanol resulting | |
|---|---|---|
| | from fermentation | Butanol resulting |
| | of renewable | from fossil |
| | starting | starting |
| | materials | materials |
| | (analysis before | (analysis after |
| | purification) | purification) |
| | (%) | (%) |
| Butanal | 0.0037 | |
| 2-Butanol | 0.0113 | <0.0010 |
| n-Butyl acetate | 0.0009 | |
| Isobutanol | 0.0662 | 0.0960 |
| n-Butanol | 99.5 | 99.8 |
| 2-Buten-1-ol | 0.1112 | |
| 1,1-Dibutoxybutane | 0.0139 | |

The n-butanol resulting from a fermentation of renewable starting materials exhibits a lower isobutanol/n--butanol ratio than purified butanol resulting from fossil starting materials, this being the case even before the optional stage of isolation of the n-butanol. Isobutanol and n-butanol exhibit very similar physiochemical properties, so that it is expensive to separate these products. The use of n-butanol which is depleted in isobutanol and in other byproducts thus constitutes a major economic advantage for the process which is a subject matter of the invention, since it makes it possible to produce butyl acrylate with a purity greater than that of an ex-petrochemical butanol BuA at a lower cost.

The use of carbon-comprising starting materials of natural and renewable origin can be detected by virtue of the carbon atoms participating in the composition of the final product. This is because, unlike the materials resulting from fossil materials, the materials composed of bioresourced renewable starting materials comprise ¹⁴C. All the samples of carbon drawn from living organisms (animal or plant organisms) are in fact a mixture of 3 isotopes: ¹²C (representing ˜98.892%), ¹³C (˜1.108%) and ¹⁴C (traces: 1.2×10⁻¹⁰%). The ¹⁴C/¹²C ratio of living tissues is identical to that of the atmosphere. In the environment, ¹⁴C exists in two predominant forms: in the inorganic form, that is to say in the form of carbon dioxide gas (CO₂), and in the organic form, that is to say in the form of carbon incorporated in organic molecules.

In a living organism, the ¹⁴C/¹²C ratio is kept constant by the metabolism because the carbon is continually exchanged with the environment. As the proportion of ¹⁴C is substantially constant in the atmosphere, it is the same in the organism, as long as it is alive, since it absorbs this ¹⁴C as it absorbs the ¹²C. The ¹⁴C/¹²C mean ratio is equal to 1.2×10⁻¹².

¹²C is stable, that is to say that the number of ¹²C atoms in a given sample is constant over time. ¹⁴C, for its part, is radioactive (each gram of carbon of a living being contains enough ¹⁴C isotope to give 13.6 disintegrations per minute) and the number of such atoms in a sample decreases over time (t) according to the law:

n=no exp(−at)

in which:
no is the ¹⁴C number at the start (at the death of the creature, animal or plant),
n is the number of ¹⁴C atoms remaining at the end of time t,
a is the disintegration constant (or radioactive constant); it is related to the half-life.

The half-life (or period) is the period of time, at the end of which any number of radioactive nuclei or of unstable particles of a given entity is reduced by half by disintegration; the half-life T_{1/2} is related to the disintegration constant a by the formula aT_{1/2}−In 2. The half-life of ¹⁴C has a value of 5730 years.

In view of the half-life (T_{1/2}) of ¹⁴C, it is considered that the ¹⁴C content is substantially constant from the extraction of the plant starting materials up to the manufacture of the final product, for example polymer, and even up to the end of its use.

The Applicant Company considers that a product or a polymer results from renewable starting materials if it comprises at least 15% (0.2×10⁻¹²/1.2×10⁻¹²) by weight of C of renewable origin with regard to the total weight of carbon, preferably at least 50% by weight of C of renewable origin with regard to the total weight of carbon.

In other words, a product or a polymer results from renewable starting material, that is to say a product or a polymer is bioresourced, if it comprises at least 0.2×10⁻¹⁰% by weight of ¹⁴C, preferably 0.6×10⁻¹⁰% by weight of ¹⁴C, with regard to the total weight of carbon. More particularly, a product or a polymer is bioresourced if it comprises from 0.2×10⁻¹⁰% to 1.2×10⁻¹⁰% by b weight of ¹⁴C.

There currently exists at least two different techniques for measuring the ¹⁴C content of a sample:
By liquid scintillation spectrometry: this method consists in counting the “β” particles resulting from the disintegration of the ¹⁴C. The β radiation resulting from a sample of known weight (known number of carbon atoms) is measured for a certain time. This “radioactivity” is proportional to the number of ¹⁴C atoms, which can thus be determined. The ¹⁴C present in the sample emits β radiation which, on contact with the liquid scintillant (scintillator), gives rise to photons. These photons have different energies (of between 0 and 156 KeV) and form what is referred to as a ¹⁴C spectrum. According to two alternative forms of this method, the analysis relates either to the CO₂ produced beforehand by combustion of the carbon-comprising sample in an appropriate absorbing solution or to the benzene after prior conversion of the carbon-comprising sample to benzene.
By mass spectrometry: the sample is reduced to graphite or to CO₂ gas and analyzed in a mass spectrometer. This technique uses an accelerator and a mass spectrometer in order to separate the ¹⁴C ions from the ¹²C ions and thus to determine the ratio of the two isotopes.

These methods for measuring the ¹⁴C content of the materials are clearly described in Standard ASTM D 6866 (in particular D6866-06) and in Standard ASTM D 7026 (in particular 7026-04). These methods compare the data measured on the analyzed sample with the data of a reference sample of 100% bioresourced origin, to give a relative percentage of bioresourced carbon in the sample. The ¹⁴C/¹²C ratio or the content by weight of ¹⁴C with respect to the total weight of carbon can subsequently be deduced therefrom for the sample analyzed.

The measurement method preferably used is the mass spectrometry described in the standard ASTM D6866-06 (“accelerator mass spectroscopy”).

The invention also targets the use of the esters comprising at least 0.2×10⁻¹⁰% by weight of ¹⁴C obtained according to the process of the invention in its various alternative forms as monomers or comonomers for the polymerization of polymer or copolymer compounds with an industrial purpose.

It also targets the polymers or copolymers manufactured from the esters synthesized according to the processes of the invention.

### EXAMPLES

The process of the invention is illustrated by the following examples.

### Example 1 (Comparative)

### Synthesis of ADAME from Petrochemical TAA

The process consists, in a first stage, in synthesizing acrolein by oxidation of propylene. This stage is carried out in the gas phase in the presence of a catalyst based on oxides of molybdenum and of bismuth, at a temperature in the vicinity of 320° C. and at atmospheric pressure. In a second stage, the acrolein-rich gaseous outlet stream resulting from the first stage is subjected to a selective oxidation reaction to give acrylic acid in the presence of molecular oxygen and of a catalyst composed of a mixed oxide of molybdenum/vanadium comprising copper and antimony, at a temperature of the order of 260° C. and at atmospheric pressure.

The reactions are carried out in laboratory fixed bed reactors. The first oxidation reactor is composed of a reaction tube with a diameter of 22 mm filled with 500 ml of catalyst for the oxidation of propylene to give acrolein and immersed in a salt bath (KNO₃, NaNO₃ and NaNO₂ eutectic mixture) maintained at a temperature of 320° C. It is fed with a gas mixture composed of 8 mol % of propylene, 8 mol % of water, air in an amount necessary in order to obtain an O₂/propylene molar ratio of 1.8/1, and nitrogen as the remainder.

The exiting gas mixture is subsequently conveyed as feed to a second reactor for the oxidation of the acrolein to give acrylic acid composed of a reaction tube with a diameter of 30 mm filled with 500 ml of catalyst and immersed in a bath of heat-exchange salt of the same type as that of the first reaction stage maintained at a temperature of 260° C.

At the outlet of the second reactor, the gas mixture is introduced at the bottom of an absorption column, countercurrentwise to a stream of water introduced at the column top. In the lower part, the column, filled with ProPack packing, is equipped with a condensation section, at the top of which a portion of the condensed mixture recovered at the column bottom is recycled, after cooling in an external exchanger.

The following phase consists in purifying the acrylic acid in order to obtain the technical acrylic acid grade. To do this, use is made of a series of successive distillations known to a person skilled in the art. The aqueous solution obtained is distilled in the presence of methyl isobutyl ketone (MIBK) solvent, which makes possible the removal of the water at the column top, after separating by settling of the heteroazeotropic MIBK/water mixture, and reflux of the solvent at the top. The dehydrated acrylic acid recovered at the column bottom is conveyed as feed to a topping column, which makes it possible to remove the light compounds, essentially acetic acid, at the top. Finally, the topped acrylic acid recovered at the bottom of this column is conveyed as feed to a tailing column, which makes it possible to remove the heavy compounds at the bottom. The acrylic acid obtained at the column top constitutes the technical acrylic acid (TAA).

In a third stage, the technical acrylic acid is esterified with ethanol in the presence of a catalyst composed of Lewatit K1461 acidic resins with the following temperature and pressure conditions: T: 80° C. and P: 1.5×10⁵ Pa. The reaction is carried out by continuously feeding the reactants (TAA, ethanol) to a first reaction step composed of 2 reactors placed in parallel comprising the resins. The stream exiting from the 1st step goes into a 2nd reaction step composed of a reactor comprising the resins. The 2 reaction steps are in series. At the inlet of the 1st step, the operation is carried out in an excess of ethanol with an ethanol/AA molar ratio of 2; at the inlet of the 2nd step, the operation is carried out in an excess of TAA by injection of TAA originating from the bottom of the first distillation column which separates the TAA from the EA/ethanol/water mixture (in this case, the TAA/ethanol molar ratio is 2). The stream at the outlet of the 2nd reaction step is purified by distillation and liquid/liquid extraction. In addition to the first column 1 mentioned above, the distillation line comprises 4 other distillation columns and a liquid/liquid extraction column.

The top product from the first column, comprising the EA/ethanol/water mixture, is conveyed to a distillation column which is used to concentrate this mixture, at the top, towards a value as close as possible to the theoretical EA/ethanol/water azeotropic mixture. A stream predominantly comprising water is recovered at the bottom of this column. The column top product is conveyed to a liquid extraction column which makes it possible to separate the EA from the ethanol/water mixture. This mixture is treated on a distillation column in order to take out:
at the top, the concentrated ethanol/water mixture, which is recycled to the reaction,
at the bottom, water, which is returned to the extraction column.

The top product from the extraction column, composed of an EA/light compound/heavy compound mixture, is conveyed to a distillation column which takes out:
at the top, the light compounds (essentially ethyl acetate),
at the bottom, the EA and the heavy compounds (furfural, various additives, such as stabilizers, and the like).

The bottom product from the column 5 is conveyed to a distillation column 6 which takes out:
at the top, the pure EA,
at the bottom, the heavy compounds.

Finally, in a final stage, the transesterification of the ethyl acrylate by the aminoalcohol of formula (CH₃)₂—N—CH₂—CH₂OH is carried out in the presence of a catalyst composed of tetraethyl titanate at a temperature of 115° C. in a stirred reactor at a pressure of 8.67×10⁴ Pa.

The furfural contents measured by UV/visible spectrophotometry in the presence of aniline (the sensitivity threshold is 0.5 ppm) during the various stages were 300 pmm in the acrolein of the first stage, 120 ppm in the TAA, 10 ppm in the ethyl acrylate and finally 3 ppm in the final ester.

### Example 2

### Synthesis of ADAME from ex-glycerol TAA

The experimentation of example 1 is reproduced while employing, as starting material during the first two stages, glycerol subjected first of all to a dehydration to give acrolein and then to an oxidation of the latter to give acrylic acid, the final two stages being identical.

The dehydration reaction is carried out in the gas phase in a fixed bed reactor in the presence of a solid catalyst composed of a tungstated zirconia ZrO₂/WO₂ at a temperature of 320° C. at atmospheric pressure. A mixture of glycerol (20% by weight) and water (80% by weight) is conveyed to an evaporator in the presence of air in an O₂/glycerol molar ratio of 0.6/1. The gas medium exiting from the evaporator at 290° C. is introduced into the reactor, consisting of a tube with a diameter of 30 mm charged with 400 ml of catalyst and immersed in a salt bath (KNO₃, NaNO₃ and NaNO₂ eutectic mixture) maintained at a temperature of 320° C. At the outlet of the reactor, the gaseous reaction mixture is conveyed to the bottom of a condensation column. This column consists of a lower section filled with Raschig rings surmounted by a condenser in which a cold heat-exchange fluid circulates. The cooling temperature in the exchangers is adjusted so as to obtain, at the column top, a temperature of the vapors of 72° C. at atmospheric pressure. Under these conditions, the loss of acrolein at the condensation column bottom is less than 5%.

This gas mixture is introduced, after addition of air (O₂/acrolein molar ratio of 0.8/1) and of nitrogen in an amount necessary in order to obtain an acrolein concentration of 6.5 mol %, as feed of the reactor for the oxidation of acrolein to give acrylic acid. This oxidation reactor consists of a tube with a diameter of 30 mm charged with 480 ml of catalyst based on Mo/V mixed oxide and immersed in a salt bath identical to that described above maintained at a temperature of 250° C. Before being introduced over the catalytic bed, the gas mixture is preheated in a tube which is also immersed in the salt bath.

At the reaction outlet, the gas mixture is subjected to a purification treatment identical to that of comparative example 1.

The 3rd and 4th stages, esterification and transesterification, are carried out under the conditions of example 1.

The furfural contents measured in the streams by UV/visible spectrophotometry during the various stages were such that the ratio by weight of furfural to acrolein was 70 ppm in the feed of the reactor for the oxidation of acrolein to give acrylic acid, after condensation of the water, 30 ppm in the TAA, 3 ppm in the ethyl acrylate and, finally, <0.5 ppm in the final ester.

These measurements of very low amounts are problematic and subject to the vagaries of the operating conditions. Much more revealing are the results obtained during the polymerization of these molecules after their quaternization. This is because the viscosity of the polymer obtained from the molecule of example 1 is 3.6 cPs, whereas that of the polymer resulting from the molecule of example 2 is 4.5 cPs, which means that the molecular weight of the latter polymer is markedly higher than that of that of example 1.

### Example 3 (Comparative)

### Synthesis of 2EHA from Petrochemical TAA

The first two stages of example 1 are repeated and the technical acrylic acid obtained after the purification stages described in example 1 is esterified with the alcohol of formula CH₃—(CH₂)₃—CH(C₂H₅)—CH₂OH under the following conditions.

The esterification reaction is carried out in the liquid phase at a temperature of 95° C. in a slight excess of TAA and in the presence of a Lewatit K2621 resin under a pressure of 0.65×10⁵ Pa.

In each of the outlet streams, the maleic anhydride content is measured by reverse phase high performance liquid chromatography. The chromatography column is a Lichrosphere 100 RP 18 with a length of 250 mm and an internal diameter of 4 mm. The eluent is a water/methanol mixture. The detector is a UV detector operating at 225 nm.

At the outlet of the first stage, the acrylic acid has a maleic anhydride content of 1% by weight. After purification, the TAA has a maleic anhydride content of 1500 ppm and, after the esterification stage and the purification by distillation following it, the acidity in the purified product is reduced to 150 ppm.

### Example 4

### Synthesis of 2EHA from ex-glycerol TAA

The first two stages of example 2 are repeated and the technical acrylic acid obtained is esterified with the alcohol of formula CH₃—(CH₂)₃—CH(C₂H₅)—CH₂OH under the conditions described in example 3.

The concentration by weight of maleic anhydride with respect to the acrolein is less than 1% by weight in the feed of the 2nd reaction stage, after condensation of the water, the content in the technical acrylic acid is of the order of 500 ppm and the final acidity in the purified 2EHA is <40 ppm.

1-22. (canceled)

23. A process for synthesizing an acrylic acid ester comprising the steps of:
a) subjecting glycerol to a dehydration reaction in the presence of an acid catalyst to form acrolein;
b) converting the acrolein using catalytic oxidation to form acrylic acid; and
c) esterifying the acrylic acid using an alcohol of formula ROH to form an acrylic acid ester of formula I: CH₂═CH—COOR, wherein R is an alkyl radical having 1 to 18 carbon atoms wherein optionally one of the carbon atoms in the alkyl radical may be replaced with a nitrogen atom.

24. The process of claim 23, wherein step a) comprises a gas phase reaction of the glycerol at a temperature ranging from 150° C. to 500° C., and a pressure ranging from 1×10⁵ Pa to 5×10⁵ Pa and in the presence of one or more solid acid catalysts having a Hammett acidity of less than +2.

25. The process of claim 23, wherein step b) comprises oxidizing the acrolein at a temperature ranging from 200° C. to 350° C., under a pressure ranging from 1×10⁵ Pa to 5×10⁵ Pa and in the presence of a solid oxidation catalyst comprising at least one element selected from Mo, V, W, Re, Cr, Mn, Fe, Co, Ni, Cu, Zn, Sn, Te, Sb, Bi, Pt, Pd, Ru or Rh, wherein the at least one element is in metallic form, or oxide, sulfate or phosphate form.

26. The process of claim 23, wherein step c) is carried out at a temperature ranging from 60° C. to 90° C. and at a pressure ranging from 1.2×10⁵ Pa to 2×10⁵ Pa and in the presence of either i) an acid catalyst in a homogeneous single-phase medium, or ii) a solid acid catalyst in a heterogeneous two-phase medium.

27. The process of claim 23, wherein step c) comprises at least two substeps comprising: i) reacting the acrylic acid and an alcohol of formula R₀OH to form an acrylic acid ester of formula II: CH₂═CH—COOR₀, wherein R₀ is selected from —CH₃, —C₂H₅, —C₃H₇, or —C₄H₉, and ii) transesterifying the acrylic acid ester of formula II to form a desired acrylic acid ester of formula I.

28. The process of claim 27, wherein the transesterification is carried out in the presence of a transesterification catalyst and at least one polymerization inhibitor at a temperature ranging from 20° C. to 120° C. and at a pressure that is equal to or lower than atmospheric, wherein the transesterification catalyst is selected from one or more of alkyl titanates, tin derivatives, zirconium derivatives, magnesium derivatives, or calcium derivatives.

29. An acrylic acid ester of formula I: CH₂═CH—COOR made by the process of claim 23, wherein R is a linear or branched alkyl radical having from 1 to 18 carbon atoms, wherein optionally one of the carbon atoms in the alkyl radical may be replaced with a nitrogen atom, and wherein the acrylic acid ester of formula I has at least 0.2×10^{<10}% by weight of ¹⁴C based on the total weight of carbon in the ester of formula I.

30. The acrylic acid ester of claim 29, wherein the alcohol ROH used in step c) is bioresourced.

31. The acrylic acid ester of claim 30, wherein the alcohol is n-butanol obtained by aerobic fermentation of biomass in the presence of bacteria.

32. A method of making a polymer or copolymer comprising using as monomers or comonomers in a polymerization reaction one or more acrylic acid esters of claim 23.

33. A polymer or copolymer made by the process of claim 32.

34. A process for synthesizing an acrylic acid ester of formula CH₂═CH—COO—CH₂—CH(C₂H₅)—(CH₂)₃—CH₃ comprising the steps of
a) subjecting glycerol to a dehydration reaction in the presence of an acid catalyst to form acrolein;
b) converting the acrolein using catalytic oxidation to form acrylic acid; and
c) esterifying the acrylic acid under acid catalysis and using an alcohol of formula CH₃—(CH₂)₃—CH(C₂H₅)—CH₂OH.

35. The process of claim 34, wherein step a) comprises a gas phase reaction at a temperature ranging from 150° C. to 500° C., and at a pressure ranging from 1×10⁵ Pa to 5×10⁵ Pa in the presence of one or more solid acid catalysts having a Hammett acidity of less than +2.

36. The process of claim 34, wherein step b) is carried out at a temperature ranging from 200° C. to 350° C., and at a pressure ranging from 1×10⁵ Pa to 5×10⁵ Pa and in the presence of a solid oxidation catalyst comprising at least one element selected from Mo, V, W, Re, Cr, Mn, Fe, Co, Ni, Cu, Zn, Sn, Te, Sb, Bi, Pt, Pd, Ru or Rh, wherein the at least one element is present in metallic form, or in oxide, sulfate or phosphate form.

37. The process of claim 34, wherein the step c) esterification is carried out at a temperature ranging from 60° C. to 90° C. and at a pressure ranging from 1.2×10⁵ Pa to 2×10⁵ Pa and either in the presence of i) an acid catalyst in a homogeneous single-phase medium, or ii) a solid acid catalyst in a heterogeneous two-phase medium.

38. An acrylic acid ester of formula CH₂═CH—COO—CH₂—CH(C₂H₅)—(CH₂)₃—CH₃ made by the process of claim 34, wherein the ester comprises at least 0.2×10⁻¹⁰% by weight of ¹⁴C, based on the total weight of carbon in the ester.

39. A process for synthesizing an acrylic acid amino ester of formula CH₂═CH—COO—CH₂—CH₂—N(CH₃)₂ comprising the steps of:
a) subjecting glycerol to a dehydration reaction in the presence of an acid catalyst to form acrolein;
b) converting the acrolein by oxidation to form acrylic acid;
c) esterifying the acrylic acid using an alcohol of formula R₀OH, wherein R₀ is selected from —CH₃,—C₂H₅, —C₃H₇, or —C₄H₉, to form an ester; and
d) transesterifying the ester formed in step c) using an amino alcohol of formula (CH₃)₂—N—CH₂—CH₂OH to form the acrylic acid amino ester.

40. The process of claim 39, wherein step a) comprises a gas phase reaction conducted at a temperature ranging from 150° C. to 500° C., and at a pressure ranging from 1×10⁵ Pa to 5×10⁵ Pa in the presence of one or more solid acid catalysts having a Hammett acidity of less than +2.

41. The process of claim 39, wherein step h) is carried out at a temperature ranging from 200° C. to 350° C., at a pressure ranging from 1×10⁵ Pa to 5×10⁵ Pa, and in the presence of a solid oxidation catalyst comprising at least one element selected from Mo, V, W, Re, Cr, Mn, Fe, Co, Ni, Cu, Zn, Sn, Te, Sb, Bi, Pt, Pd, Ru or Rh, wherein the at least one element is present in metallic form, or in oxide, sulfate or phosphate form.

42. The process of claim 39, wherein R₀ of step c) is selected from —CH₃, —C₂H₅, —C₃H₇ or —C₄H₉, and wherein the esterification of step c) is conducted at a temperature ranging from 60° C. to 90° C., at a pressure ranging from 1.2×10⁵ Pa to 2×10⁵ Pa and in the presence of either i) an acid catalyst in a homogeneous single-phase medium, or ii) a solid acid catalyst in a heterogeneous two-phase medium.

43. The process of claim 39, wherein the transesterification of step d) is carried out in the presence of a transesterification catalyst and at least one polymerization inhibitor at a temperature ranging from 20° C. to 120° C., at a pressure that is equal to or lower than atmospheric pressure, wherein the transesterification catalyst is selected from one or more of alkyl titanates, tin derivatives, zirconium derivatives, magnesium derivatives, or calcium derivatives.

44. An acrylic acid amino ester of formula CH₂═CH—COO—CH₂—CH₂—N(CH₃)₂ made by the process of claim 39, wherein the acrylic acid amino ester comprises at least 0.2×10⁻¹⁰% by weight of ¹⁴C, based on the total weight of carbon in the acrylic acid amino ester.
